(19) United States
(12) Patent Application Publication
(10) Pub. No.: US 2010/0094321 A1
(43) Pub. Date: Apr. 15, 2010

(54) Ultrasound Handpiece

(76) Inventors: Takayuki Akahoshi, Tokyo (JP); Mikhail Boukhny, Laguna Niguel, CA (US)

Correspondence Address: ALCON, IP LEGAL, TB4-8, 6201 SOUTH FREEWAY, FORT WORTH, TX, 76134, US

(21) Appl. No.: 12/249,329

(22) Filed: Oct. 10, 2008

(51) Int. Cl. A61B 17/32

(52) U.S. Cl. 606/169

(57) ABSTRACT

A handpiece having at least one pair of vibrating piezoelectric elements that are polarized and arranged within the handpiece so as to produce vibrations that are orthogonal, or otherwise at an angle to, the longitudinal centerline of the handpiece. Vibrating the cutting tip at an angle to its longitudinal axis creates additional shearing at the distal end of the tip, enhancing the tissue cutting action of the tip.

This invention relates to ultrasonic devices and more particularly to an ophthalmic phacoemulsification handpieces.

### BACKGROUND OF THE INVENTION

A typical ultrasonic surgical device suitable for ophthalmic procedures consists of an ultrasonically driven handpiece, an attached hollow cutting tip, an irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece at its nodal points by relatively inflexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve.

When used to perform phacoemulsification, the ends of the cutting tip and irrigating sleeve are inserted into a small incision of predetermined width in the cornea, sclera, or other location in the eye tissue in order to gain access to the anterior chamber of the eye. The cutting tip is ultrasonically vibrated along its longitudinal axis within the irrigating sleeve by the crystal-driven ultrasonic horn, thereby emulsifying upon contact the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the bore of the cutting tip, the horn bore, and the aspiration line and into a collection device. The aspiration of emulsified tissue is aided by a saline flushing solution or irrigant that is injected into the surgical site through the small annular gap between the inside surface of the irrigating sleeve and the outside surface of the cutting tip.

There have been prior attempts to combine ultrasonic longitudinal motion of the cutting tip with rotational or oscillating motion of the tip, see U.S. Pat. No. 5,222,959 (Anis), U.S. Pat. No. 5,722,945 (Anis, et al.) and U.S. Pat. No. 4,504,264 (Kelman), the entire contents of which are incorporated herein by reference. These prior attempts have used electric motors to provide the rotation of the tip which require O-ring or other seals that can fail in addition to the added complexity and possible failure of the motors. All electronic handpieces using specially polarized piezoelectric crystals are described in U.S. Pat. No. 6,077,285 (Boukhny), and one commercially available handpiece, the OZIL™ handpiece, available for Alcon Laboratories, Inc., Fort Worth, Tex. is an all electronic handpiece that produces both longitudinal and torsional or twisting vibrations by the use of a series of diagonal slits in the ultrasound horn in combination with two pair of longitudinally polarized piezoelectric crystals. While this handpiece has been commercially successful, a handpiece that produces additional shearing motions is desirable.

Accordingly, a need continues to exist for a simple, reliable ultrasonic handpiece that produces high shearing forces at the distal end of the handpiece cutting tip.

### BRIEF SUMMARY OF THE INVENTION

The present invention improves upon prior art ultrasonic devices by providing a handpiece having at least one pair of vibrating piezoelectric elements that are polarized and arranged within the handpiece so as to produce vibrations that are orthogonal, or otherwise at an angle to, the longitudinal centerline of the handpiece. Vibrating the cutting tip at an angle to its longitudinal axis creates additional shearing at the distal end of the tip, enhancing the tissue cutting action of the tip.

It is accordingly an object of the present invention to provide an ultrasound handpiece having enhances cutting action.

It is a further object of the present invention to provide an ultrasound handpiece having ultrasonic vibrations that are at an angle to the longitudinal axis of the handpiece.

Other objectives, features and advantages of the present invention will become apparent with reference to the drawings, and the following description of the drawings and claims.

### BRIEF DESCRIPTION OF THE DRAWINGS

FIG. 1 is a perspective view of a prior art ultrasound handpiece.

FIG. 2 is a partial cross-section view of a prior art ultrasound handpiece.

FIG. 3 is a partial cross-section view of one embodiment of the ultrasound handpiece of the present invention.

FIG. 4 is a schematic illustration of the movement in one embodiment of the ultrasound handpiece of the present invention.

FIG. 5 a graphical representation of the movement in one embodiment of the ultrasound handpiece of the present invention.

### DETAILED DESCRIPTION OF THE INVENTION

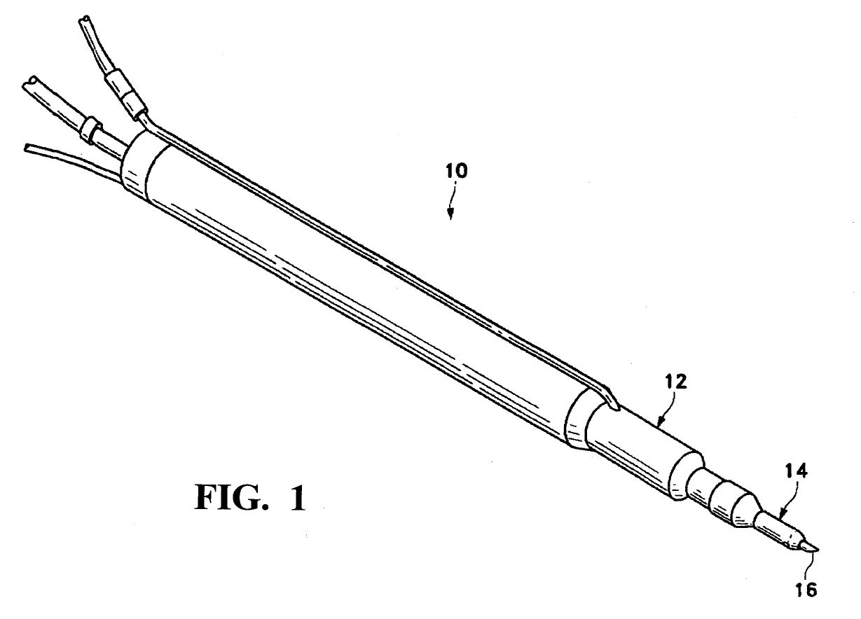

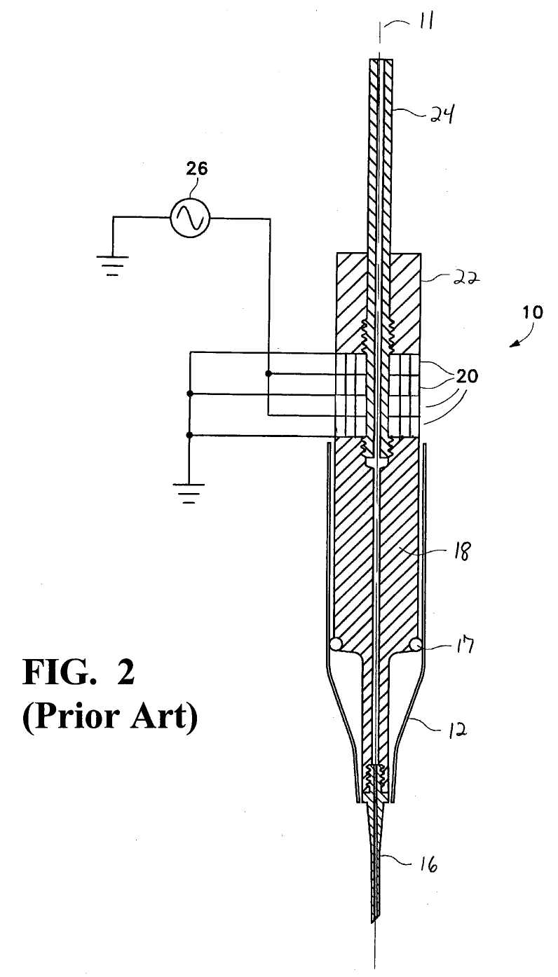

As best seen in FIGS. 1 and 2, prior art handpiece 10 generally includes handpiece shell 12, infusion sleeve 14 and cutting tip 16. As best seen in FIG. 2, handpiece 10 contains ultrasonic horn 18. Handpiece 10 generally contains two one pair of piezoelectric crystals 20 coupled to horn 18 so as to create longitudinal vibratory motion in horn 18 when the crystals are excited. As used herein, the term “longitudinal” means parallel to logitudinal centerline 11. The materials used for and the construction of handpiece 10 and horn 18 are well-known to those skilled in the art. See, for example, U.S. Pat. No. 4,169,984 (Parisi), U.S. Pat. No. 4,515,583 (Sorich), U.S. Pat. No. 4,989,588 (Kubota, et al.) and U.S. Pat. No. 5,359,996 (Hood), the entire contents of which being incorporated herein by reference. Horn 18 is held within shell 12 by isolator 17. Crystals 20 are held within shell 12 and in contact with horn 18 by back cylinder 22 and bolt 24. Crystals 20 vibrate ultrasonically in response to a signal generated by ultrasound generator 26.

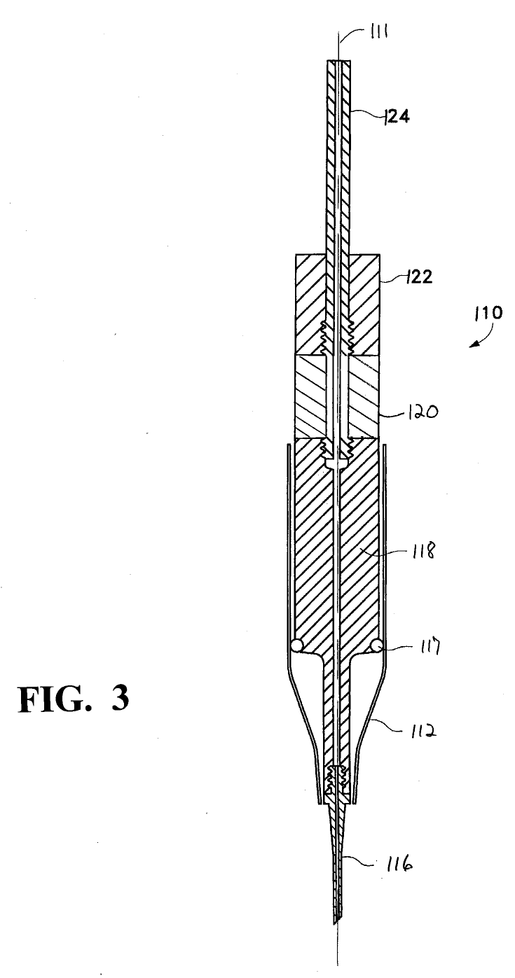

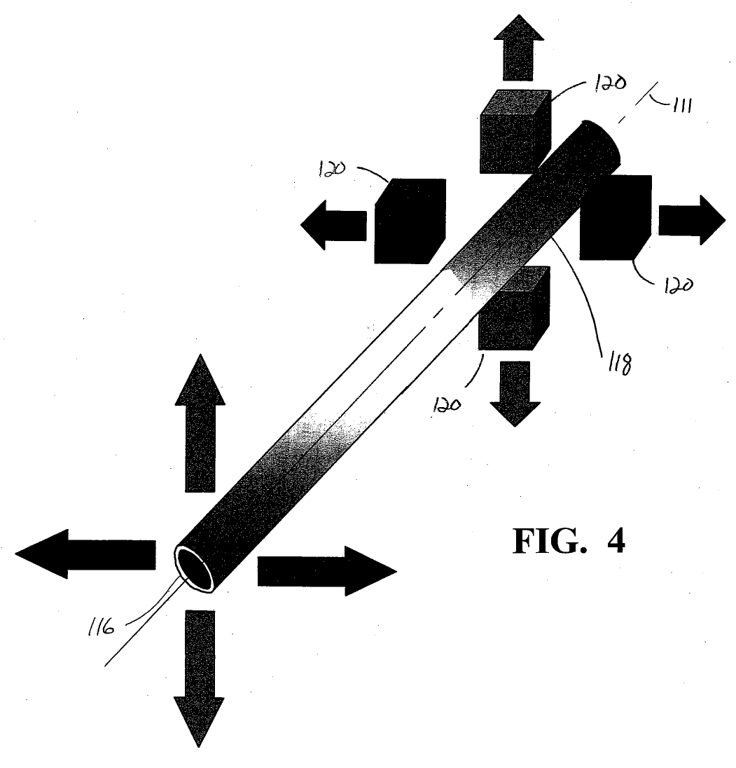

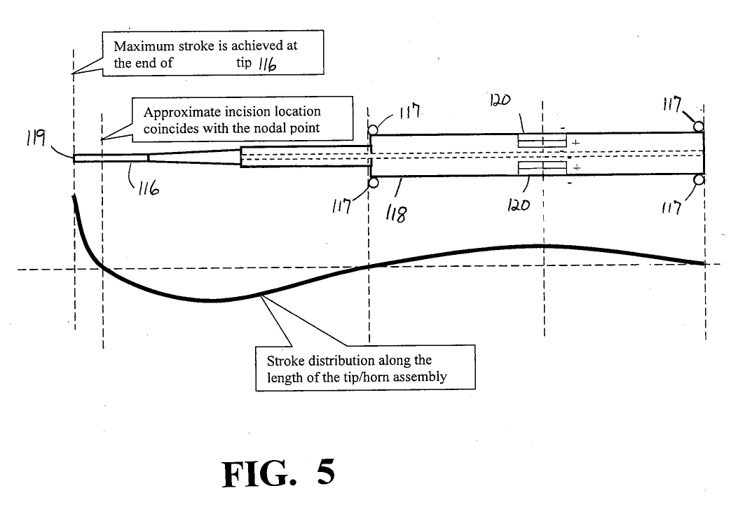

As best seen in FIGS. 3, 4 and 5, handpiece 110 of the present invention generally includes handpiece shell 112, infusion sleeve 114 and cutting tip 116. Handpiece 110 is similar in outward appearance as handpiece 10 illustrated in FIG. 1. As best seen in FIG. 4, handpiece 110 contains ultrasonic horn 118. Handpiece 110 generally contains two one pair of piezoelectric crystals 120 coupled to horn 118 so as to create vibratory motion in horn 118 that are at an angle to centerline 111, preferably a right angle or orthogonal. Horn 118 is held within shell 112 by isolators 117. Crystals 120 are held within shell 112 and in contact with horn 118 by back cylinder 122 and bolt 124. While one set of crystals 120 can produce vibrations at one angle relative to centerline 111, additional set(s) of crystals 120 can produce vibrations at different angles and/or along different orthogonal planes relative to centerline 111, either alternatively or simultaneously, as seen in FIG. 4. As shown in FIG. 5, locating isolators 117 at the nodal points of horn 118 causes the point of maximum orthogonal deflection to be located at distal end 119 of tip 116.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. An ultrasound surgical handpiece, comprising:
a) a shell;
b) an ultrasonic horn held within the shell;
c) a tip connected to a distal end of the horn, the horn and the tip have a longitudinal centerline entending down the length of the horn and tip; and
d) at least one pair of piezoelectric crystals connected to the ultrasonic horn opposite the tip, the piezoelectric crystals arranged so at to produce ultrasonic motion in the horn and tip that is at an angle relative the the longitudinal centerline.

2. The handpiece of claim 1 wherein the angle is a right angle.

3. The handpiece of claim 1 wherein the horn is located within the shell so as to produce a point of maximum orthogonal deflection located at a distal end of the tip.

4. The handpiece of claim 1 further comprising a plurality of sets of piezoelectric crystals, each set of crystals producing vibrations along different planes orthogonal to the longitudinal centerline.

5. An ultrasound surgical handpiece tip, comprising:
a) an ultrasonic horn held within the shell;
c) a tip connected to a distal end of the horn, the horn and the tip have a longitudinal centerline entending down the length of the horn and tip; and
d) a plurality of piezoelectric crystals connected to the ultrasonic horn opposite the tip, the piezoelectric crystals arranged so at to produce a point of maximum orthogonal deflection located at a distal end of the tip, each set of crystals producing vibrations along different planes orthogonal to the longitudinal centerline.
